(19) United States
(12) Patent Application Publication
(10) Pub. No.: US 2012/0035600 A1
(43) Pub. Date: Feb. 9, 2012

(54) BIOLOGICAL TISSUE TRANSFORMATION USING ULTRAFAST LIGHT

(76) Inventors: David Gaudiosi, Rohnert Park, CA (US); Carolyn Martinez, Petaluma, CA (US); Michael Armas, Petaluma, CA (US); Michael Mielke, Santa Rosa, CA (US); Michael Greenberg, Santa Rosa, CA (US); Tim Booth, Petaluma, CA (US)

(21) Appl. No.: 13/204,540

(22) Filed: Aug. 5, 2011

Related U.S. Application Data
(60) Provisional application No. 61/371,641, filed on Aug. 6, 2010.

(51) Int. Cl. A61B 18/20

(52) U.S. Cl. 606/10; 606/13

(57) ABSTRACT

Systems and methods for transforming biological materials utilizing ultrafast laser light. According to some embodiments, a method for transforming a biological material may include calculating an ablation profile for the biological material by comparing initial characteristics of the biological material to desired characteristics for the biological material, and applying an output of an ultrafast laser to the biological material to transform the biological material using the ablation profile in such a way that collateral damage to remaining biological material is reduced.

### CROSS-REFERENCE TO RELATED APPLICATION

This non-provisional patent application claims the priority benefit of U.S. provisional patent application number 61/371,641, filed on Aug. 6, 2010, entitled “BIOLOGICAL TISSUE TRANSFORMATION USING ULTRAFAST LIGHT”—which is hereby incorporated by reference in its entirety.

### FIELD OF THE TECHNOLOGY

The present technology relates generally to systems and methods for transforming biological material, and more specifically, but not by way of limitation, to systems and methods that utilize ultrafast laser pulses to ablate a surface of a biological material without causing collateral damage to remaining biological material.

### BACKGROUND

According to some embodiments, the present technology transforms biological tissue, e.g. mammalian skin, by using ultrafast light, e.g. from an ultrafast (ultrashort pulse) laser light source. The biological tissue may be transformed by way of altering its shape, dimensions, texture, uniformity, morphology, mass, color, softness, hardness, porosity, transparency, density, or any of its macroscopic or microscopic properties. There are numerous motivations and applications for performing these transformations including (but not limited to) therapies to treat specific physiologies, preparing tissue for subsequent steps in a larger procedure, and sectioning and sculpting tissue for implant or other treatment of a medical patient.

Additionally, in some embodiments, the transformation of the biological tissue may occur via ultrafast laser ablation processes (e.g., cold ablation), which minimize or substantially reduce collateral damage to remaining and/or surrounding biological tissue.

### SUMMARY OF THE TECHNOLOGY

According to some embodiments, the present technology may be directed to methods for transforming a biological material. These methods may include: (a) calculating an ablation profile for the biological material by comparing initial characteristics of the biological material to desired characteristics for the biological material; and (b) applying ultrafast laser pulses from an ultrafast laser to the biological material to transform the biological material according to the ablation profile in such a way that collateral damage to remaining biological material is reduced.

In other embodiments, the present technology may be directed to additional methods for transforming a biological material that include (a) calculating an ablation height for a section of the biological material relative to a same section of a desired profile of the biological material; and (b) applying ultrafast laser output of an ultrafast laser to the section to remove an amount of material from the section that is substantially equal to the ablation height, wherein collateral damage to remaining biological material of the section is substantially reduced.

In additional embodiments, the present technology may be directed to systems for transforming material that include (a) a memory for storing executable instructions for transforming materials; (b) a processor for executing the instructions, the instructions comprising: (i) a biological material evaluation module that determines initial characteristics of the biological material; (ii) an ablation profile generator that calculates an ablation profile for the biological material by comparing the initial characteristics of the biological material to desired characteristics for the biological material; and (iii) a laser controller module that causes an ultrafast laser to output ultrafast laser pulses that transform the biological material according to the ablation profile, in such a way that collateral damage to remaining the biological material is substantially reduced.

### BRIEF DESCRIPTION OF THE DRAWINGS

FIG. 1 is a block diagram of an exemplary ultrafast laser.

FIG. 2 is a block diagram of an exemplary ultrafast laser control system application for controlling the ultrafast laser of FIG. 1.

FIG. 3A is an illustration of an exemplary material surface profile cross-section.

FIG. 3B is an illustration of an exemplary material surface profile cross-section after ablation.

FIGS. 4 and 5 illustrate examples of biological materials that are ablated in a particular region and non-ablated in another region.

FIG. 6 illustrates an example of an unablated tissue.

FIG. 7 illustrates an example of an ablated tissue.

FIG. 8 illustrates a table of laser device parameter combinations.

FIG. 9 illustrates a table of tissue thickness data.

FIG. 10 illustrates an exemplary computing system that may be used to implement embodiments according to the present technology.

### DETAILED DESCRIPTION

The present technology transforms biological tissue, e.g. mammalian skin, by using ultrafast light, e.g. from an ultrafast (ultrashort pulse) laser light source. The biological tissue may be transformed by way of altering its shape, dimensions, texture, uniformity, morphology, mass, color, softness, hardness, porosity, transparency, density, or any of its macroscopic or microscopic properties. There are numerous motivations and applications for performing these transformations including (but not limited to) therapies to treat specific physiologies, preparing tissue for subsequent steps in a larger procedure, and sectioning and sculpting tissue for implant or other treatment of a medical patient.

In some exemplary applications, the present technology may be used for surface structuring via selective ultrashort pulse “USP” laser ablation. In many technologies and industries, there exists a need for the preparation of surfaces or interfaces having a prescribed surface profile (planar, specific spherical curvature, saddle shape, etc.) or thickness profile. In some cases, the required surface morphology is difficult to create due to physical characteristics of the biological material or the lack of a suitable technology for shaping, forming, polishing, or otherwise modifying the surface of the biological material whose profile must be carefully controlled. To cite just one example from the medical industry, skin grafting requires the preparation of donor skin that has uniform thickness and smooth surface so that the resulting graft heals properly and the resulting graft has a natural appearance. Since donor skin is in very limited supply, it is necessary to obtain the highest yield of suitable graft material from the donor skin. This requires very thin sectioning of the donor skin. Skin grafts that are improperly prepared may have an irregular thickness or surface morphology and may be unsuitable for use.

As such, USP lasers as provided herein, are advantageous tools that selectively ablate the surface of many types of biological materials. These USP lasers can be focused onto a small section of a biological material. Additionally, the size and shape of the ablation zone can be controlled by adjusting the spot size, the pulse energy level, the laser repetition rate, and the number of pulses arriving at any specific location on the surface of a biological material.

The present technology may be performed by an USP laser which ablates the surface of a biological material. The USP lasers provided herein may be configured to ablate a wide range of biological materials, such as hard tissues (e.g. bone, cartilage, and so forth) and soft tissues such as skin. Additionally, one of ordinary skill in the art will recognize the suitability of the USP laser for milling, drilling, boring, cutting, sectioning, shaving, or otherwise transforming many types of biological materials.

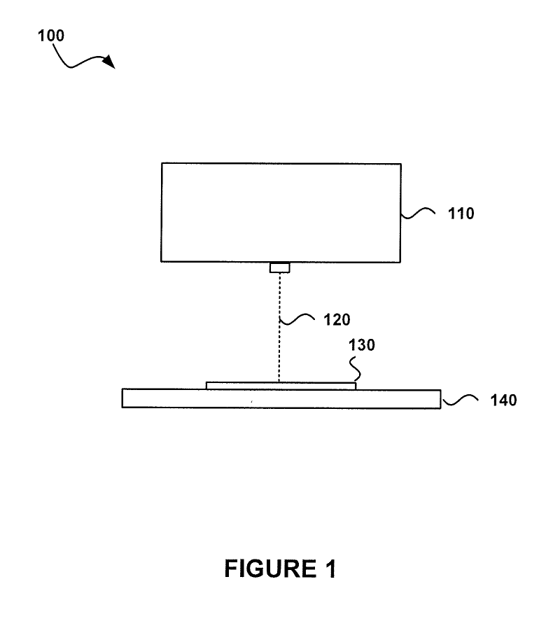

FIG. 1 is a block diagram of an exemplary ultrafast laser system 100. The system 100 includes an ultrafast laser device, hereinafter “Laser assembly 110,” a target biological tissue (material) 130, and a platform 140. Laser assembly 110 may direct pulses of ultrafast laser light 120 at target biological tissue 130, which rests on platform 140. In some embodiments, parameters of the Laser assembly 110 may be adjusted as the laser light is directed at tissue 130. According to some embodiments, the Laser assembly 110 may include a ultrafast laser emitting sub-assembly that generates ultrafast laser output and a beam delivery sub-assembly (not shown) that includes a combination of mirrors, mounts, and/or other required optical or structural components for directing the delivery of the ultrafast laser output of the USP laser emitter sub-assembly.

In some embodiments, the platform 140 translates relative to the Laser assembly 110, and in other embodiments, the Laser assembly 110 is translated relative to the platform 140. In yet other embodiments, both the Laser assembly 110 and the platform 140 may be translated relative to each another. Additionally, in some embodiments, rather than translating the entire Laser assembly 110 and/or the platform 140, the beam delivery sub-assembly may be selectively adjusted to deliver energy pulses from the laser emitting sub-assembly to different sections of the biological tissue disposed on the platform 140.

Generally speaking, the Laser assembly 110 may include any laser emitting device that is configured to deliver energy in short pulses to ablate material. It is noteworthy to mention that the strength of the electric field generated by the beam of the USP laser may increase significantly (above the ablation point of the biological material) such that the target molecules of the material begin to be ionized and create a plasma, in a process known as optical breakdown. Ultimately, these molecules are removed from the material surface, or “ablated”, without collateral damage to the remaining biological material. In contrast to the nanosecond and picosecond USP lasers provided herein, long pulse devices utilize energy waves for ablating material that cause a mix of optical breakdown and traditional thermal processes. Unfortunately, these long pulse systems cause significant heat transfers to the biological material and can thermally damage collateral areas surrounding the focal point of the beam. This effect is particularly deleterious when the target material includes biological tissues, which may be susceptible to thermal damage that may render the biological tissue non-viable for use.

USP lasers produce pulses of light that may be less than a picosecond long in duration. The shortness of the pulses ensures that the ablation process is substantially caused by optical breakdown, while minimizing or eliminating thermal breakdown or degradation. Therefore, precise features may be machined into a variety of materials without introducing heat or thermal irregularities to the sample material.

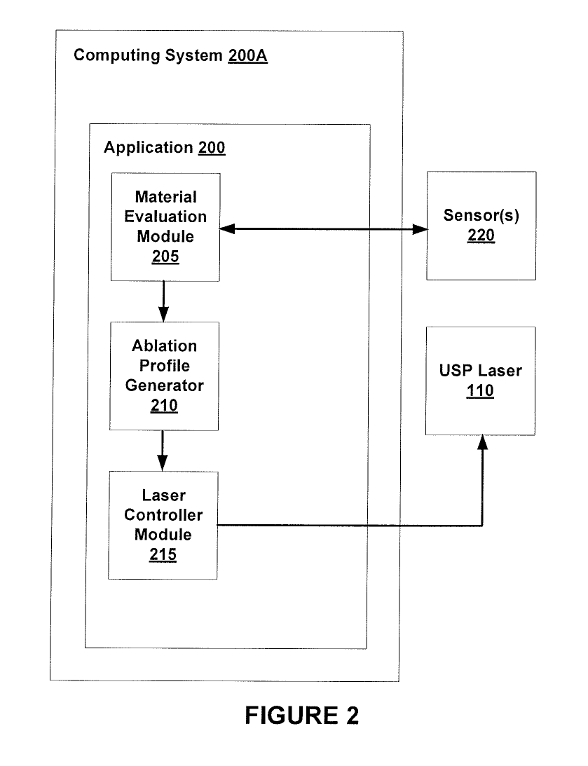

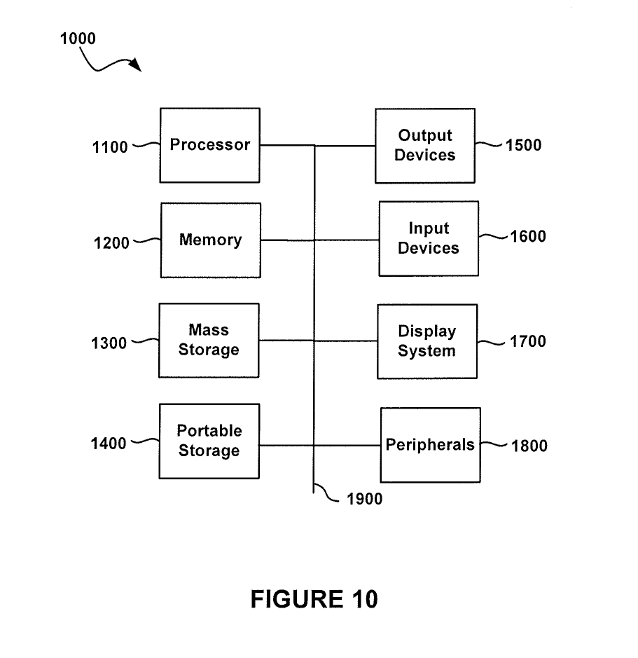

FIG. 2 illustrates a block diagram of an exemplary ultrafast laser control system application, hereinafter referred to as “application 200.” The application 200 may be embodied in executable instructions that are stored in memory and executable by a processor of a computing system 200A to cause a USP laser to transform a biological material. Computing system 200A may be generally described with reference to computing system 1000 illustrated in FIG. 10.

According to some embodiments, the application 200 may generally include a biological material evaluation module 205, an ablation profile generator 210, and a laser controller module 215. It is noteworthy that the application 200 may include additional modules, engines, or components, and still fall within the scope of the present technology. As used herein, the term “module” may also refer to any of an application-specific integrated circuit (“ASIC”), an electronic circuit, a processor (shared, dedicated, or group) that executes one or more software or firmware programs, a combinational logic circuit, and/or other suitable components that provide the described functionality.

The biological material evaluation module 205 may be configured to determine initial characteristics of the biological tissue. Initial characteristics may include an initial or “first” profile of the surface of the biological material, a density of the biological material, an ablation point for the biological material, a moisture content for the biological material, and so forth. These initial characteristics may be utilized to selectively adjust parameters of the Laser assembly 110 for efficacious ablation of the biological material.

The biological material evaluation module 205 may be communicatively coupled with one or more sensors 220 that measure one or more of the characteristics of the biological material, as described above. One of ordinary skill in the art would be well versed in selecting an appropriate sensor for measuring each of the different characteristics of the biological material; therefore a detailed discussion of suitable sensors will be omitted for the purposes of brevity.

In some embodiments, the biological material evaluation module 205 may utilize optical coherence topography or laser position sensor scanning processes, or any other measurement processes that would be known to one of ordinary skill in the art with the present disclosure before them to determine the profile of the surface of the biological material. In other embodiments, physical measurement processes such as compression measurement utilizing a dial indicator may be employed. It will be understood that, mechanical thickness measurements determined by compression may yield different results relative to uncompressed optical measurements. Additionally, a surface profile for the biological material may be determined by the calculation of Moire fringes (e.g., patterns) for the surface of biological material.

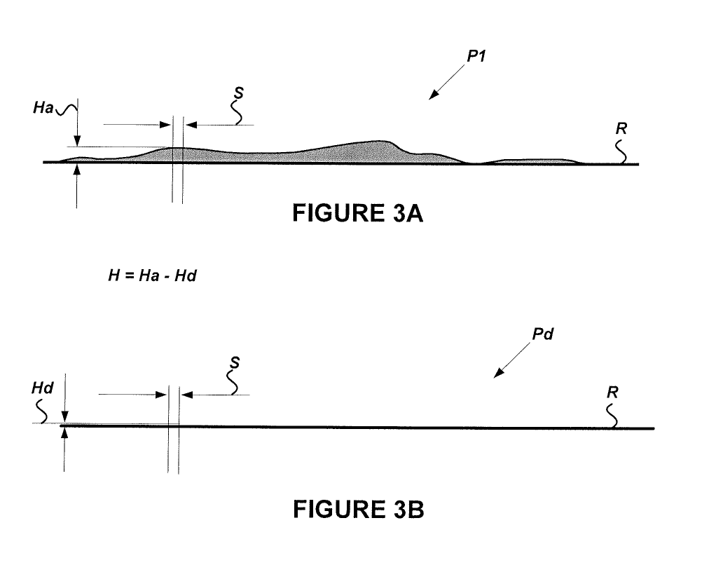

Referring now to FIGS. 2, 3A and 3B collectively, in some embodiments, the biological material evaluation module 205 may calculate an ablation height H (not depicted in the figures for clarity) of a given section S of the biological material relative to a horizontal reference plane R. That is, the biological material may be mapped or divided into any number of sections S, and an ablation height H may be calculated for each section S. In other embodiments, rather than a reference plane R, the ablation height H may be calculated by comparing the actual height Ha of the section to a desired height Hd for the same section of a desired profile Pd. The ablation height H may be understood include a difference between the actual height Ha and the desired height Hd of the section S.

It is noteworthy to mention that in FIG. 3B the desired height Hd for the desired profile Pd is substantially equal to zero relative to the reference plane R. Indeed, in this example, the desired height Hd for each section of the desired profile Pd is substantially equal to zero relative to the reference plane R, which indicates that the desired profile Pd is substantially flat.

According to some embodiments, an ablation height H for a section S may be determined by comparing a first profile P1 to a second profile, which in this example is represented by the desired profile Pd.

Utilizing one or more of the above-described methods, the ablation profile generator 210 may generate an ablation profile for the biological material. The ablation profile may be utilized by the laser controller module 215 to determine parameters for the ultrafast laser. Generally speaking, the ablation profile may be utilized by the laser controller module 215 similarly to a computer-aided design that is utilized by a computer numeric controlled machine to machine (e.g., mill, rout, drill, and so forth) a biological material.

In operation, the ablation profile may specify that one or more sections require more ablation relative to other adjacent sections. Based upon this data, the laser controller module 215 may selectively adjust the parameters of the ultrafast laser to ablate the desired amount of material from the section (substantially equal to the ablation height) in such a way that collateral damage to the remaining biological material is substantially reduced or eliminated.

It will be understood that a particularized set of parameters for the Laser assembly 110 (based upon the ablation profile of the biological material) may be referred to as the USP laser or “ultrafast laser” profile.

Parameters of the USP laser may include, but are not limited to, a pattern (e.g. traversal path of the platform 140 relative to the USP laser), a pulse speed, a focal point, a fill spacing, number of passes, and combinations thereof. The focal point of the USP laser may be understood to include a distance between the end of the Laser assembly 110 and the point at which the USP laser output is focused to deliver the selected ablation energy pulses. Fill spacing may include the distance between ablated rows (and may be measured from the centerline of a row to the centerline of an adjacent row). A pulse speed may be understood to include a distance, transverse to the propagation direction, traveled by a focal point of the laser energy pulses in a given period of time.

Each of these parameters may be selectively adjusted based upon the initial characteristics of the biological material (e.g., material properties) and the ablation profile for the biological material. For example, for a section having an ablation height that is greater relative to the ablation height of another section, the focal point of the USP laser may be selectively increased after each successive ablation energy pulse. In other examples, the pulse speed may be decreased to deliver more ablation pulses in a section to remove more material from a section relative to a higher pulse speed.

The biological material properties of the biological material may also dictate the pulse speed, energy level, and other USP laser parameters. For example, the energy level required to ablate soft biological tissue may have a magnitude that is lower relative to energy levels utilized to ablate hard biological tissues such as bone.

The sensors 220 communicatively coupled with the biological material evaluation module 205 may periodically or continually evaluate the profile of the biological material to determine if the parameters of the USP laser are transforming the biological material according to the ablation profile. Comparisons may be made between an actual ablated profile (an actual amount of ablation) and an expected ablation profile (an amount of ablation expected based upon the characteristics of the biological material). If deviations are detected, the laser controller module 215 may selectively adjust one or more of the parameters of the Laser assembly 110 to accommodate for the detected deviations. For example, if an insufficient amount of material has been removed from a section of the biological material, the laser controller module 215 may decrease the pulse speed or increase the energy level of the Laser assembly 110 to increase the amount of material removed.

It will be understood that deviations may arise from imperfections, defects or unexpected heterogeneity of the biological material. Additionally, the moisture content of the biological material may also affect the efficiency of the Laser assembly 110. As such, one or more of the sensors 220 may be configured to determine the moisture content of the biological material. Based upon the moisture content and the ablation profile, the system 100 may apply a predetermined amount of fluid to the biological material. Advantageously, any one of a number of fluids may be utilized such as water, saline, oils, and so forth.

In sum, based upon feedback gathered by the sensors 220, the laser controller module 215 may dynamically and selectively adjust the parameters of the Laser assembly 110 to ensure that the Laser assembly 110 produces a final material that substantially conforms to the desired profile.

According to some embodiments the desired profile may include any two-dimensional or three-dimensional geometrical configurations. Therefore, the final shape of the biological material may be finely contoured or tailored for specific uses. For example, a section of a hard tissue such as acetabulum of a human pelvis may be reshaped to matingly couple with an implantable medical device that is inserted into the acetabulum, such as an acetabuluar cup. Ablation of the surface of the bone to create a pattern on the surface of the acetabulum allows for adhesive disposed between the medical device and the acetabulum to engage more surface area of the bone, improving the mechanical bond.

In an exemplary application of the present technology, incoming samples (10) were large rectangular pieces of tissue, approximately 70 mm×90 mm. The objective of this work was to demonstrate the ability to mill the tissue to thinner dimensions.

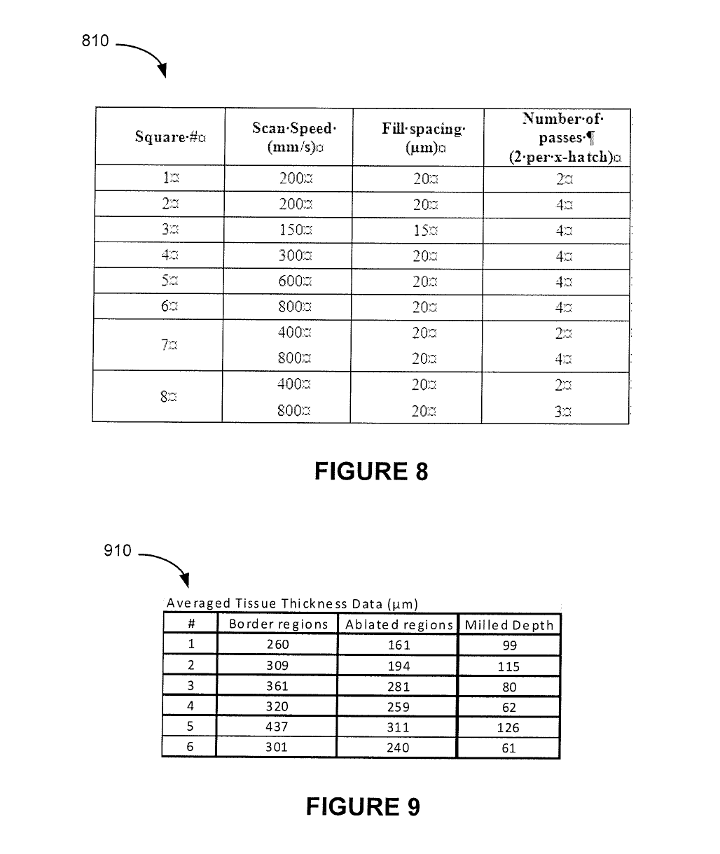

A 35 mm×50 mm area of tissue was milled using a pattern including 1 cross hatch cycle of coarse milling and 2 cross hatch cycles for fine milling (Table of FIG. 8).

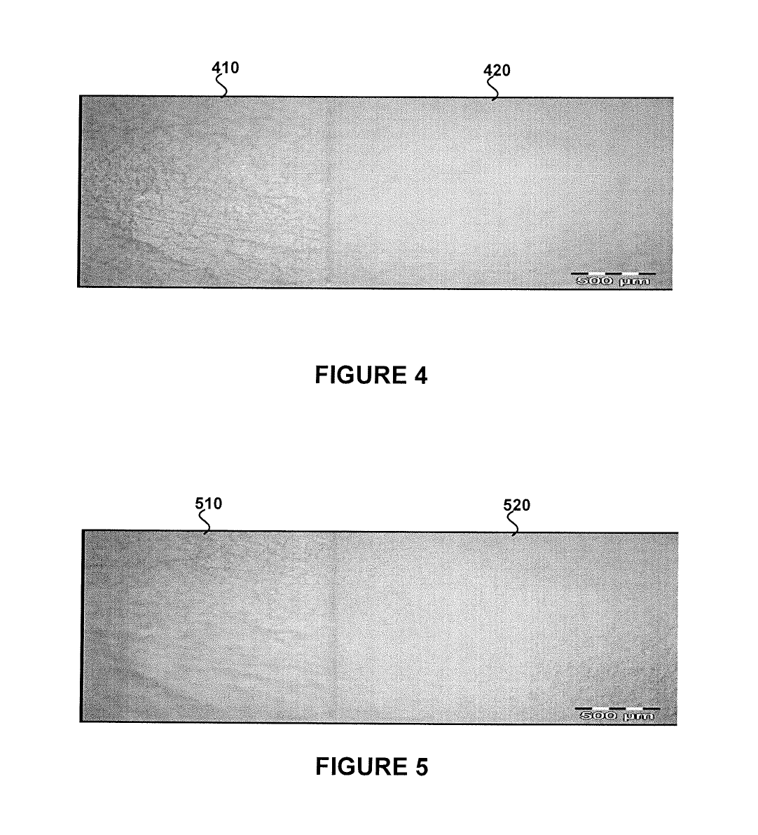

Two 35 mm×50 mm areas were milled on each large tissue sample with no evidence of thermal damage. The milled areas were noticeably smoother in texture than the areas that were not ablated (see FIGS. 4 and 5).

FIGS. 4 and 5 include photographs of the same sample of biological tissue. FIG. 4 is focused on a higher plane of the un-ablated biological material. The left section 410 of the photograph shows the un-ablated region before ablation, and the right section 420 of the photograph shows the milled features of the biological material with show no signs of thermal damage—the biological material is shown as having clean, smooth edges. FIG. 5 shows the same sample as FIG. 4, but was instead focused on the milled area and shows a difference in the ablated texture and coloration of the tissue in the right section 520 relative to the left section 510 of the photograph.

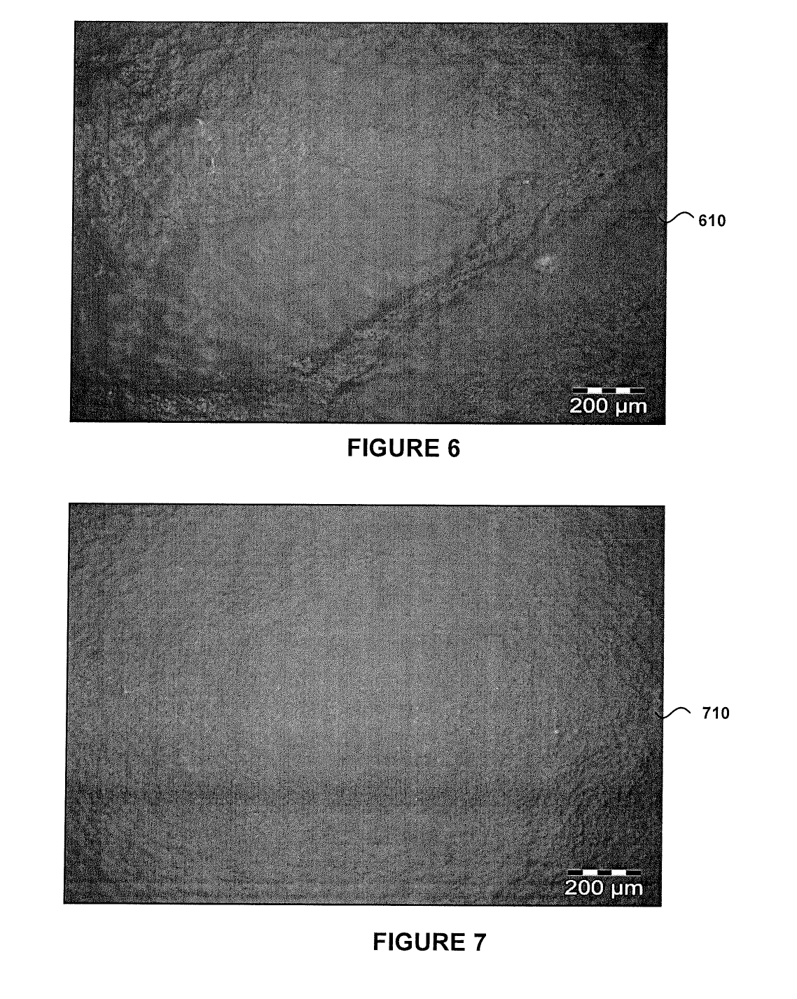

FIGS. 6 and 7: FIG. 6 illustrates a photograph of un-ablated material 610. FIG. 7 illustrates the same material 710 after ablation with ultrafast laser pulses from a USP laser constructed in accordance with the present technology.

The ultrafast laser used to mill porcine tissue samples in the exemplary cases includes a commercially available laser, such as, for example, an ultrafast laser characterized by a full width at half maximum (FWHM) temporal intensity duration of less than one picosecond, a pulse energy up to 50 microJoules, and a repetition rate up to 100 kHz. However, any suitable laser may be used with the present technology.

The pulse speed, fill spacing and number of passes were varied in seven sections to demonstrate the process development (Table of FIG. 8).

A random sample was used to determine the base reference plane for the ablation. All the samples were placed in this plane regardless of the tissue thickness. Thus tissue which was thicker than the first sample was milled to a deeper depth while tissues which were thinner were not milled as deeply.

The Table of FIG. 9 contains the averaged data for the six tissue samples with two milled areas on each. The tissue thickness was measured with a dial indicator, which compresses the tissue. Thus, the compressed thickness measurements are different from the uncompressed optical measurements.

An exemplary embodiment of the Laser assembly 110 may include the following laser profile having one or more of the parameters listed below:

### Processing Parameters

System: A USP laser characterized by a FWHM temporal intensity duration less than one picosecond, a pulse energy up to 50 microJoules, and a repetition rate up to 100 kHz.
Beam delivery: Dual axis scanning lens, galvanometer scanner, 2× beam expander
Laser spot size: 30 μm
Lens focal length: 112 mm
Polarization state: elliptical
Process gas: none

### Laser Parameters

Pulse energy: 31.5 μJ (on target)
Average power: 1.6 W
Repetition rate: 50 kHz
Pulse width: 650 fs (not measured)

### Motion Parameters

Scanner speed: Coarse milling speed 400 mm/s (20 μm fill spacing in a cross hatch pattern)
Fine milling speed 800 mm/s (20 μm fill spacing in a cross hatch pattern)
Part process time: Milling 1651.7 seconds for an area approximately 35 mm×50 mm

### Processed Sample

Feature dimensions: 35 mm×50 mm
Depth=330 μm
   Cleaning Method: none

According to some embodiments, a texture can be transferred into some materials, such as biological tissue, by placing a textured substrate in direct contact with an unablated side of the biological material and performing a multi-pass milling process (e.g., applying ultrafast laser output) to thin the tissue. The use of a textured substrate may produce effects in the biological material that appear analogous to material effects caused by copper rubbing. Copper rubbing produces a pattern in a thin material by pressing of the copper material into a rigid textured surface.

In the case of the tissue, a shock wave caused by the ultrafast laser output during the ablation may result in the exertion of a force (e.g., pressure) that causes a surface of the biological material to be pressed into the fine structure of the substrate. This pressing may cause local density changes that result in a change of ablation rate which is then transferred into the tissue.

Additionally, increases in tissue conformity result in a temporary change in the surface profile of the biological material during ablation. This change in the surface profile is then made permanent by milling the irregular surface flat. The texture is revealed upon removal of pressure applied to thereto.

FIG. 10 illustrates an exemplary computing system 1000 that may be used to implement an embodiment of the present technology. The system 1000 of FIG. 10 may be implemented in the contexts of the likes of computing systems, networks, servers, or combinations thereof. The computing system 1000 of FIG. 10 includes one or more processors 1100 and main memory 1200. Main memory 1200 stores, in part, instructions and data for execution by processor 1100. Main memory 1200 may store the executable code when in operation. The system 1000 of FIG. 10 further includes a mass storage device 1300, portable storage medium drive(s) 1400, output devices 1500, user input devices 1600, a graphics display 1700, and peripheral devices 1800.

The components shown in FIG. 10 are depicted as being connected via a single bus 1900. The components may be connected through one or more data transport means. Processor unit 1100 and main memory 1200 may be connected via a local microprocessor bus, and the mass storage device 1300, peripheral device(s) 1800, portable storage device 1400, and display system 1700 may be connected via one or more input/output (I/O) buses.

Mass storage device 1300, which may be implemented with a magnetic disk drive or an optical disk drive, is a non-volatile storage device for storing data and instructions for use by processor unit 1100. Mass storage device 1300 may store the system software for implementing embodiments of the present invention for purposes of loading that software into main memory 1200.

Portable storage device 1400 operates in conjunction with a portable non-volatile storage medium, such as a floppy disk, compact disk, digital video disc, or USB storage device, to input and output data and code to and from the computer system 1000 of FIG. 10. The system software for implementing embodiments of the present invention may be stored on such a portable medium and input to the computer system 1000 via the portable storage device 1400.

Input devices 1600 provide a portion of a user interface. Input devices 1600 may include an alphanumeric keypad, such as a keyboard, for inputting alpha-numeric and other information, or a pointing device, such as a mouse, a trackball, stylus, or cursor direction keys. Additionally, the system 1000 as shown in FIG. 10 includes output devices 1500. Suitable output devices include speakers, printers, network interfaces, and monitors.

Display system 1700 may include a liquid crystal display (LCD) or other suitable display device. Display system 1700 receives textual and graphical information, and processes the information for output to the display device.

Peripherals 1800 may include any type of computer support device to add additional functionality to the computer system. Peripheral device(s) 1800 may include a modem or a router.

The components provided in the computer system 1000 of FIG. 10 are those typically found in computer systems that may be suitable for use with embodiments of the present invention and are intended to represent a broad category of such computer components that are well known in the art. Thus, the computer system 1000 of FIG. 10 may be a personal computer, hand held computing system, telephone, mobile computing system, workstation, server, minicomputer, mainframe computer, or any other computing system. The computer may also include different bus configurations, networked platforms, multi-processor platforms, etc. Various operating systems may be used including Unix, Linux, Windows, Macintosh OS, Palm OS, Android, iPhone OS and other suitable operating systems.

It is noteworthy that any hardware platform suitable for performing the processing described herein is suitable for use with the technology. Computer-readable storage media refer to any medium or media that participate in providing instructions to a central processing unit (CPU), a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD-ROM disk, digital video disk (DVD), any other optical storage medium, RAM, PROM, EPROM, a FLASHEPROM, any other memory chip or cartridge.

While various embodiments have been described above, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

What is claimed is:

1. A method for transforming a biological material, comprising:
calculating an ablation profile for the biological material by:
comparing initial characteristics of the biological material to desired characteristics for the biological material; and
applying ultrafast laser pulses from an ultrafast laser to the biological material to transform the biological material according to the ablation profile in such a way that collateral damage to remaining biological material is reduced.

2. The method according to claim 1, wherein calculating an ablation profile includes:
determining an initial surface profile for the section;
determining material properties for the biological material; and
calculating the ablation profile from the initial surface profile and the biological material properties relative to a desired surface profile for the biological material.

3. The method according to claim 1, further comprising calculating an ultrafast laser profile for the ultrafast laser based upon the ablation profile of the biological material.

4. The method according to claim 3, wherein the ultrafast laser profile includes any of a pattern, a pulse speed, pulse energy level, a focal point, laser repetition rate, a fill spacing, number of passes, and combinations thereof, for the ultrafast laser.

5. The method according to claim 4, further comprising:
determining an intermediate surface profile for the biological material during the step of applying ultrafast laser output of an ultrafast laser to the biological material;
comparing the intermediate surface profile for the biological material to an expected surface profile, the expected surface profile being estimated from the ablation profile of the biological material and the ultrafast laser profile; and
automatically adjusting the ultrafast laser profile of the ultrafast laser based upon a difference between the intermediate surface profile and the expected surface profile to change the ultrafast laser pulses from the ultrafast laser.

6. The method according to claim 1, further comprising determining moisture content of the biological material, and applying a predetermined amount of fluid to the biological material during the step of applying ultrafast laser output of an ultrafast laser to the biological material based upon the moisture content.

7. The method according to claim 1, wherein desired characteristics of the biological material include a desired shape, the desired shape including at least one of:
a two dimensional geometric configuration; and
a three dimensional geometrical configuration.

8. The method according to claim 1, further comprising translating the biological material relative to the ultrafast laser, based upon the ablation profile of the biological material.

9. The method according to claim 1, wherein determining an initial surface profile for the section includes the utilization of optical coherence topography.

10. The method according to claim 1, wherein determining an initial surface profile for the section includes the utilization of laser position sensor scanning.

11. The method according to claim 1, wherein determining an initial surface profile for the section includes the calculation of Moire fringes for a surface of the biological material.

12. A method for precision milling of a biological material, comprising:
calculating an ablation height for a section of the biological material relative to a same section of a desired profile of the biological material; and
applying ultrafast laser output of an ultrafast laser to the section to remove an amount of material from the section that is substantially equal to the ablation height, wherein collateral damage to remaining biological material of the section is substantially reduced.

13. The method according to claim 12, further comprising:
translating the biological material relative to the ultrafast laser; and
adjusting one or more ultrafast laser parameters of the ultrafast laser during the step of translating based upon the ablation height calculated for a current section.

14. The method according to claim 13, wherein the ultrafast laser parameters include at least one of a pulse speed, pulse energy level, and a focal point for the ultrafast laser.

15. The method according to claim 12, wherein additional ultrafast laser parameters include any of a pattern, a fill spacing, laser repetition rate, number of passes, and combinations thereof.

16. The method according to claim 12, wherein the ablation height for the section of the biological material is calculated relative to a reference plane that extends at least partially through the biological material.

17. The method according to claim 12, further comprising applying a textured substrate to a first surface of the biological material and applying ultrafast laser output of an ultrafast laser to a second surface of the biological material to impart a texture to the first surface.

18. A system for transforming a biological material, comprising:
a memory for storing executable instructions for transforming biological materials;
a processor for executing the instructions, the instructions comprising:
a biological material evaluation module that determines initial characteristics of the biological material;
an ablation profile generator that calculates an ablation profile for the biological material by:
comparing the initial characteristics of the biological material to desired characteristics for the biological material; and
a laser controller module that causes an ultrafast laser to output ultrafast laser pulses that transform the biological material according to the ablation profile, in such a way that collateral damage to the biological material is substantially reduced.

19. The system according to claim 18, wherein the ablation profile generator is further configured to:
determine an initial surface profile for the section;
determine material properties for the biological material; and
calculate the ablation profile from the initial surface profile and the biological material properties relative to a desired surface profile for the biological material.

20. The system according to claim 18, wherein the laser controller module calculates an ultrafast laser profile for the ultrafast laser based upon the ablation profile of the biological material.
